(19) United States
(12) Patent Application Publication
(10) Pub. No.: US 2024/0011006 A1
(43) Pub. Date: Jan. 11, 2024

(54) METHODS AND MEANS OF INACTIVATING RNA BASED VACCINES

(71) Applicant: James VELTMEYER, La Jolla, CA (US)

(72) Inventor: James VELTMEYER, La Jolla, CA (US)

(21) Appl. No.: 18/349,030

(22) Filed: Jul. 7, 2023

Related U.S. Application Data
(60) Provisional application No. 63/359,280, filed on Jul. 8, 2022.

(51) Int. Cl. C12N 9/22; A61K 47/69

(52) U.S. Cl. CPC C12N 9/22; A61K 47/6909

(57) ABSTRACT

The invention provides methods of inactivating biological activity of RNA based therapeutics, such as vaccines, through administration of compositions possessing or stimulating RNAse activity alone or together with lipid based formulations capable of delivering RNAse or RNAse stimulating activity into said RNAse based therapeutic.

### CROSS-REFERENCE TO RELATED APPLICATIONS

This application claims priority to U.S. Provisional Application No. 63/359,280, titled “Methods and Means of Inactivating RNA Based Vaccines”, and filed Jul. 8, 2022, which is incorporated by reference in its entirety.

### FIELD OF THE INVENTION

The teachings herein are directed to compositions and methods for inactivating RNA based vaccines utilizing RNAse.

### BACKGROUND

Despite promising initial results there remains a population of patients which develop severe adverse reactions to RNA based therapeutics. Classical immunology teaches that vaccine-induced activation of adaptive immunity occurs weeks after immunogen stimulation. Paradoxically adverse reactions within hours or days have been reported with mRNA-based COVID-19 vaccines, implying existence of an exaggerated and potentially fatal innate immune overactivation. To date there are no means of neutralizing vaccine hyperimmune activity post administration.

### SUMMARY

Preferred embodiments are directed to methods of inactivating an RNA based therapeutic comprising administering a composition possessing RNAse activity and/or having ability to stimulate RNAse activity.

Preferred methods include embodiments wherein one or more agents are added to said composition possessing RNAse activity and/or having ability to stimulate RNAse activity, wherein said agents increase interaction between said composition possessing RNAse activity and/or having ability to stimulate RNAse activity and said RNA based therapeutic.

Preferred methods include embodiments wherein said RNA based therapeutic comprises of a mRNA molecule or plurality of molecules capable of inducing translation of one or more peptides or proteins.

Preferred methods include embodiments wherein said mRNA induces translation of one or more immunogens.

Preferred methods include embodiments wherein said immunogens are SARS-CoV-2 associated peptides or proteins.

Preferred methods include embodiments wherein said SARS-CoV-2 associated peptides or proteins are derived from the spike domain.

Preferred methods include embodiments wherein said composition possessing RNAse activity is lactoribonuclease.

Preferred methods include embodiments wherein said lactoribonuclease is isolated from mammalian milk.

Preferred methods include embodiments wherein said lactoribonuclease is isolated from bovine milk.

Preferred methods include embodiments wherein said bovine milk derived lactoribonuclease is isolated based on a molecular weight of 36.8 kDa.

Preferred methods include embodiments wherein said composition possessing RNAse activity is bacterial RNAse.

Preferred methods include embodiments wherein said composition possessing RNAse activity is yeast derived RNAse.

Preferred methods include embodiments wherein said yeast derived RNAse is isolated based on a molecular weight of 25 kDa.

Preferred methods include embodiments wherein said agent or agents capable of increasing the interaction between said composition possessing RNAse activity and/or having ability to stimulate RNAse activity and said RNA based therapeutic is a lipid.

Preferred methods include embodiments wherein said lipid is incorporated into a micelle or micelle-like composition.

Preferred methods include embodiments wherein said micelle or micelle-like composition is capable of delivering said RNAse or RNAse-inducing composition to said RNA-based therapeutic.

Preferred methods include embodiments wherein said micelle or micelle=like structure is composed of 1-palmitoyl-2-oleoyl-sn-glycerol-3-phosphocholine (POPC), and/or dimethyldioctadecylammonium bromide (DDAB), distearoylphosphatidylethanolamine-PEG2000

Preferred embodiments include methods of inhibiting an immunological hyperreaction to an mRNA vaccine comprising administration of a TLR3 antagonist.

Preferred methods include embodiments wherein said TLR3 antagonist is Poly IC.

Preferred methods include embodiments wherein said RNAse is Ranpirnase.

### DETAILED DESCRIPTION OF THE INVENTION

The invention provides means of inhibiting the ability of a mRNA vaccine to induce immune hyperactivation through, intra alia, suppression of its ability to act as a TLR3 agonist. In one embodiment of the invention administration of compositions containing RNAse activity and/or compositions possessing ability to induce RNAse activity are administered. In one embodiment said composition is RNAse obtained from bovine milk by the process of chromatographic extraction of proteins possessing a molecular weight of 36.8 kDA. In another embodiment recombinant RNAse protein obtained from commercially available sources is administered alone or together with lipids capable of facilitating contact between said RNAse and administered RNA containing therapeutic.

As used herein, the term “RNA” is used generally to refer to any molecule comprising ribose nucleotides, including fully polymerized RNAs, as well as any oligionucleotide fragment thereof, and encompasses the various types of RNA known as mRNA, rRNA, tRNA and hnRNA.

As used herein, the term “DNA” is used generally to refer to any molecule comprising deoxyribose nucleotides, including fully polymerized DNAs, as well as any oligionucleotide fragment thereof.

As used herein, the terms “ribonuclease” and “RNase” are used interchangeably to refer to those nucleases that degrade RNA into its respective constituent nucleotides or fragments. Examples of such RNases include RNase A, RNase H, RNase I, RNase T1, RNase III, as well as mixtures of these RNases.

As used herein, the term “lyophilized” refers to any composition that has been freeze-dried, typically from an aqueous solution.

For the practice of the invention a description of RNases that are useful in accomplishing inactivation of RNA therapeutics is provided. It is known that RNAses are proteolytic enzymes that selectively degrade RNA molecules, but generally does not recognize or degrade DNA molecules. Native RNases can be isolated from various cellular sources. Alternatively, the gene encoding the RNase can be isolated, amplified, and transferred to a host genome, propagated under conditions that promote production of the RNase, and then isolated using a series of standard protein purification steps. For example, the RNase isoform known as RNase T1 can be isolated from an over-expressing E. coli strain containing the cloned Aspergillus oryzae RNase T1-encoding gene. RNase T1 cleaves RNA after G residues and frequently is used for RNA mapping, as well as for some ribonuclease protection assay protocols. In addition to RNase T1, there are other isoforms of RNase, including those known as RNase A, RNase I, RNase H, etc. RNase A cleaves RNA after the C and U residues. RNase I can degrade any RNA to a mixture of mono-, di-, and trinucleotides and has a marked preference for single-stranded RNA over double-stranded RNA, which allows it to work well in methods for analyzing RNA structure or abundance. While RNase I does not degrade DNA, it will bind to it. RNase H hydrolyzes RNA only in RNA:DNA hybrids, and will not degrade single-stranded DNA or RNA. The invention teaches the use of various RNAses to inactivate and/or reduce toxicity of RNA therapeutics. RNases are widely used by molecular biologists to remove contaminating RNA molecules from DNA isolates, and for controlled proteolytic analysis of RNA structure and function. Because each RNase has a different specificity for hydrolyzing RNA, they are thus useful for different applications. For example, RNase A is most commonly used to remove RNA that is contaminating plasmid preparations, as well as for digestion of unhybridized RNA in ribonuclease protection assays. However, digestion of RNA with RNase A alone can leave fragments of RNA which are large enough to be visible on agarose gels and to precipitate in ethanol. RNase H is commonly used to destroy an RNA template after first-strand cDNA synthesis. RNase mixtures can be used where it is desirable to degrade the RNA more thoroughly such as plasmid minipreps. For example, because RNase T1 cleaves RNA after the G residues, while RNase A cleaves RNA after C and U residues, mixtures of RNase T1 and RNase A can do better job of reducing RNA fragment size over the use of either RNase alone. Indeed, plasmid DNA isolation protocols frequently require the use of RNase mixtures to degrade contaminating RNA molecules as completely as possible.

The invention, in some embodiments, the invention teaches the application of RNAse to induce Immunological tolerance to the RNA based therapeutic. It is known that a cardinal feature of the immune system, is allowing for recognition and elimination of pathological threats, while selectively ignoring antigens that belong to the body. Traditionally, autoimmune conditions or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia. In contrast to conventional approaches targeting induced immunity, the current invention aims to inactivate the immunogenic mass through selectively cleaving the immunogen encoding RNA.

In one embodiment of the invention, exosomes are utilized to deliver RNAse(s) to the biological site affected by the RNA therapeutic. The exosomes of the present invention may be targeted to a desired cell type or tissue. This targeting is achieved by expressing on the surface of the exosome a targeting moiety which binds to a cell surface moiety expressed on the surface of the cell to be targeted. Typically the targeting moiety is a peptide which is expressed as a fusion protein with a transmembrane protein typically expressed on the surface of the exosome. In more detail, the exosomes of the invention can be targeted to particular cell types or tissues by expressing on their surface a targeting moiety such as a peptide. Suitable peptides are those which bind to cell surface moieties such as receptors or their ligands found on the cell surface of the cell to be targeted. Examples of suitable targeting moieties are short peptides, scFv and complete proteins, so long as the targeting moiety can be expressed on the surface of the exosome and does not interfere with insertion of the membrane protein into the exosome. Typically the targeting peptide is heterologous to the transmembrane exosomal protein. Peptide targeting moieties may typically be less than 100 amino acids in length, for example less than 50 amino acids in length, less than 30 amino acids in length, to a minimum length of 10, 5 or 3 amino acids.

Targeting moieties can be selected to target particular tissue types such as muscle, brain, liver, pancreas and lung for example, or to target a diseased tissue such as a tumour. In a particularly preferred embodiment of the present invention, the exosomes are targeted to brain tissue. Specific examples of targeting moieties include muscle specific peptide, discovered by phage display, to target skeletal muscle, a 29 amino acid fragment of Rabies virus glycoprotein that binds to the acetylcholine receptor or a fragment of neural growth factor that targets its receptor to target neurons, the peptide transportan, and secretin peptide that binds to the secretin receptor can be used to target biliary and pancreatic epithelia. As an alternative, immunoglobulins and their derivatives, including scFv antibody fragments can also be expressed as a fusion protein to target specific antigens, such as VEGFR for cancer gene therapy. As an alternative, natural ligands for receptors can be expressed as fusion proteins to confer specificity, such as NGF which binds NGFR and confers neuron-specific targeting. The peptide targeting moiety is expressed on the surface of the exosome by expressing it as a fusion protein with an exosomal transmembrane protein. A number of proteins are known to be associated with exosomes; that is they are incorporated into the exosome as it is formed. The preferred proteins for use in targeting the exosomes of the present invention are those which are transmembrane proteins. Examples include but are not limited to Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta and tetraspanins. In particularly preferred embodiments of the present invention, the exosomes loaded with biotherapeutic protein and/or peptide are targeted using a transmembrane protein selected from Lamp-1, Lamp-2, CD13, CD86, Flotillin, Syntaxin-3. In a particularly preferred embodiment the transmembrane protein is Lamp-2. The following section relates to general features of all polypeptides, and in particular to variations, alterations, modifications or derivatisations of amino acid sequence. It will be understood that such variations, alterations, modifications or derivatisations of polypeptides as are described herein are subject to the requirement that the polypeptides retain any further required activity or characteristic as may be specified subsequent sections of this disclosure. Variants of polypeptides may be defined by particular levels of amino acid identity which are described in more detail in subsequent sections of this disclosure. Amino acid identity may be calculated using any suitable algorithm. For example the PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands. The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate homology (for example used on its default settings) (Devereux et al (1984) Nucleic Acids Research 12, 387-395). It will be understood that variants of polypeptides also includes substitution variants. Substitution variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. The amino acid sequence of polypeptides for use in the invention may be modified to include non-naturally occurring chemistries or to increase the stability and targeting specificity of the compound. When the polypeptides are produced by synthetic means, such amino acids may be introduced during production. The polypeptides may also be modified following either synthetic or recombinant production. A number of side chain modifications are known in the art and may be made to the side chains of the polypeptides, subject to the polypeptides retaining any further required activity or characteristic as may be specified herein. Variant polypeptides as described in this section are those for which the amino acid sequence varies from that in SEQ ID NO: 1, but which retain the ability to be inserted into the membrane of an exosome. The variant sequences typically differ by at least 1, 2, 3, 5, 10, 20, 30, 50, 100 or more mutations (which may be substitutions, deletions or insertions of amino acids). For example, from 1 to 100, 2 to 50, 3 to 30 or 5 to 20 amino acid substitutions, deletions or insertions may be made, provided the modified polypeptide is inserted into the membrane of an exosome.

The constructs of the invention may be administered by any suitable means. Administration to a human or animal subject may be selected from parenteral, intramuscular, intracerebral, intravascular (including intravenous), subcutaneous, intranasal, intracardiac, intracerebroventricular, intraperitoneal or transdermal administration. Typically the method of delivery is by injection. Preferably the injection is intramuscular or intravascular (e.g. intravenous). A physician will be able to determine the required route of administration for each particular patient. The constructs are preferably delivered as a composition. The composition may be formulated for any suitable means of administration, including parenteral, intramuscular, intracerebral, intravascular (including intravenous), intracardiac, intracerebroventricular, intraperitoneal, subcutaneous, intranasal or transdermal administration. Compositions for parenteral administration may include sterile aqueous solutions which may also contain buffers, diluents and other suitable additives. The constructs of the invention may be formulated in a pharmaceutical composition, which may include pharmaceutically acceptable carriers, thickeners, diluents, buffers, preservatives, and other pharmaceutically acceptable carriers or excipients and the like in addition to the exosomes. A “pharmaceutically acceptable carrier” (excipient) is a pharmaceutically acceptable solvent, suspending agent or any other pharmacologically inert vehicle for delivering one or more nucleic acids to a subject. Typical pharmaceutically acceptable carriers include, but are not limited to, binding agents (e.g. pregelatinised maize starch, polyvinylpyrrolidone or hydroxypropyl methylcellulose, etc); fillers (e.g. lactose and other sugars, microcrystalline cellulose, pectin, gelatin, calcium sulfate, ethyl cellulose, polyacrylates or calcium hydrogen phosphate, etc); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc); or wetting agents (e.g. sodium lauryl sulphate, etc). The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavouring agents, preservatives, antioxidants, opacifiers, thickening agents and stabilizers. However, such materials, when added, should not unduly interfere with the biological activities of the components of the compositions provided herein. A therapeutically effective amount of composition is administered. The dose may be determined according to various parameters, especially according to the severity of the condition, age, and weight of the patient to be treated; the route of administration; and the required regimen.

The pharmaceutical composition in accordance with the present disclosure may be formulated in the form of oral preparations such as powders, granules, capsules, tablets, aqueous suspensions, external preparations, suppositories and sterilized injection solutions according to a conventional method. The present disclosure is not limited thereto. The pharmaceutical composition in accordance with the present disclosure may include pharmaceutically acceptable carriers. The pharmaceutically acceptable carrier may include a binder, a lubricant, a disintegrant, an excipient, a solubilizing agent, a dispersing agent, a stabilizer, a suspending agent, a pigment, a perfume, etc., upon oral administration. In the case of an injectable preparation, the pharmaceutically acceptable carrier may include a buffer, a preservative, an anhydrous agent, a solubilizer, an isotonic agent, a stabilizer, etc. In the case of topical administration, the pharmaceutically acceptable carrier may include a base, an excipient, a lubricant, a preservative, etc. Formulations of the pharmaceutical composition in accordance with the present disclosure may be produced in a variety of ways, mixed with a pharmaceutically acceptable carrier as described above. For example, when administered orally, it may be produced in the form of tablets, troches, capsules, elixirs, suspensions, syrups, wafers, etc. In the case of injections, it may be produced in a unit dose ampoule or multiple doses. It may be produced in the forms of other solutions, suspensions, tablets, capsules, sustained-release preparations and the like.

In embodiment of the invention a patient previously administered an RNA based vaccine is provided a concentration of Ranpirnase in order to degrade the RNA in said vaccine. In one embodiment said Ranpirnase is administered at dose levels ranging from 60 mug/m2 (anticipated human dose) to 960 mug/m2.

In another embodiments suppression of spike protein induced toxicity is provided by administration of regenerative cells. In one embodiment said regenerative cells are stem cells. In a specific embodiment said stem cells are mesenchymal stem cells. Said stem cells are in a preferred embodiment umbilical cord tissue derived mesenchymal stem cells. In one embodiment said stem cells are administered intravenously at a concentration of 1 million to 300 million cells. Said administration may be performed at the time of vaccination or subsequent to vaccination. Further, examples of carriers, excipients and diluents suitable for formulation include lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, mineral oil, or the like. Further, fillers, anti-coagulants, lubricants, wetting agents, perfumes, emulsifiers, preservatives and the like may be further included. The administration route of the pharmaceutical composition according to the present disclosure is not limited to following but includes oral, intravenous, intramuscular, intraarterial, intramedullary, intradural, intracardiac, transdermal, subcutaneous, intraperitoneal, intranasal, topical, sublingual or rectal. Oral or parenteral administration is preferred. As used herein, “parenteral” includes subcutaneous, intradermal, intravenous, intramuscular, intraarticular, intrasynovial, intrasternal, intrathecal, intralesional and intracranial injection or infusion techniques. The pharmaceutical composition in accordance with the present disclosure may be further administered in the form of suppositories for rectal administration. The pharmaceutical composition in accordance with the present disclosure can vary widely depending on various factors including the activity of the specific compound used, age, body weight, and conventional health, gender, formula, administration time, administration route, excretion rate, drug combination and prevention or severity of specific disease to be treated. The amount of administration of the pharmaceutical composition will depend on the patient's condition, weight, severity of disease, drug form, administration route and duration, but may be appropriately selected by one skilled in the art and may be injected at a content of 0.0001 to 50 mg/kg or 0.001 to 50 mg/kg per day. Administration may be done once a day, or several sessions a day. The amount of administration does not in any way limit the scope of the present disclosure. The pharmaceutical composition according to the present disclosure may be formulated into pills, caplets, capsules, liquids, gels, syrups, slurries, and suspensions.

A physician will be able to determine the required route of administration and dosage for any particular patient. Optimum dosages may vary depending on the relative potency of individual constructs, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection. Preferably, the dose of a single intramuscular injection is in the range of about 5 to 20 .mu.g. Preferably, the dose of single or multiple systemic injections is in the range of 10 to 100 mg/kg of body weight. Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

1. A method of inactivating an RNA based therapeutic comprising administering a composition possessing RNAse activity and/or having ability to stimulate RNAse activity.

2. The method of claim 1, wherein one or more agents are added to said composition possessing RNAse activity and/or having ability to stimulate RNAse activity, wherein said agents increase interaction between said composition possessing RNAse activity and/or having ability to stimulate RNAse activity and said RNA based therapeutic.

3. The method of claim 1, wherein said RNA based therapeutic comprises of a mRNA molecule or plurality of molecules capable of inducing translation of one or more peptides or proteins.

4. The method of claim 3, wherein said mRNA induces translation of one or more immunogens.

5. The method of claim 4, wherein said immunogens are SARS-CoV-2 associated peptides or proteins.

6. The method of claim 5, wherein said SARS-CoV-2 associated peptides or proteins are derived from the spike domain.

7. The method of claim 1, wherein said composition possessing RNAse activity is lactoribonuclease.

8. The method of claim 7, wherein said lactoribonuclease is isolated from mammalian milk.

9. The method of claim 8, wherein said lactoribonuclease is isolated from bovine milk.

10. The method of claim 9, wherein said bovine milk derived lactoribonuclease is isolated based on a molecular weight of 36.8 kDa.

11. The method of claim 1, wherein said composition possessing RNAse activity is bacterial RNAse.

12. The method of claim 1, wherein said composition possessing RNAse activity is yeast derived RNAse.

13. The method of claim 12, wherein said yeast derived RNAse is isolated based on a molecular weight of 25 kDa.

14. The method of claim 1, wherein said agent or agents capable of increasing the interaction between said composition possessing RNAse activity and/or having ability to stimulate RNAse activity and said RNA based therapeutic is a lipid.

15. The method of claim 14, wherein said lipid is incorporated into a micelle or micelle-like composition.

16. The method of claim 15, wherein said micelle or micelle-like composition is capable of delivering said RNAse or RNAse-inducing composition to said RNA-based therapeutic.

17. The method of claim 15, wherein said micelle or micelle=like structure is composed of 1-palmitoyl-2-oleoyl-sn-glycerol-3-phosphocholine (POPC), and/or dimethyldioctadecylammonium bromide (DDAB), distearoylphosphatidylethanolamine-PEG2000.

18. A method of inhibiting an immunological hyperreaction to an mRNA vaccine comprising administration of a TLR3 antagonist.

19. The method of claim 18, wherein said TLR3 antagonist is Poly IC.

20. The method of claim 1, wherein said RNAse is Ranpirnase.
